(19) United States
(12) Patent Application Publication
(10) Pub. No.: US 2023/0405305 A1
(43) Pub. Date: Dec. 21, 2023

(54) ATTACHMENT MECHANISM FOR ALIGNMENT GARMENT FOR USE WITH A FULLY IMPLANTABLE SYSTEM

(71) Applicant: Medtronic, Inc., Minneapolis, MN (US)

(72) Inventors: Stephen M. Nelson, Plymouth, MN (US); David J. Peichel, Minneapolis, MN (US); Eric A. Schilling, Ham Lake, MN (US); Nathanael O. Mason, Holliston, MA (US); Robert Scadding Moir, Cambridge (GB); Lillian E. Clarke, Cambridge (GB); Ismail Akram, Cambridge (GB); Glenn Le Faou, Hertfordshire (GB); Richard James Andrews, Cambridge (GB); Jordi Vilanova Hanusaik, Cambridge (GB); Anna Rebecca Louise Wilson, Cambridge (GB); Arron Carmo do Rosario Rodrigues, Cambridge (GB)

(21) Appl. No.: 18/251,342

(22) PCT Filed: Sep. 17, 2021
(86) PCT No.: PCT/US2021/050829

§ 371 Date: May 1, 2023

Related U.S. Application Data
(60) Provisional application No. 63/108,625, filed on Nov. 2, 2020.

(51) Int. Cl. A61M 60/875

(52) U.S. Cl. CPC A61M 60/875; A61M 2209/088; A61M 2205/8243

(57) ABSTRACT

An attachment mechanism securable to a garment for holding an external coil in a predetermined location, the attachment mechanism comprising: a body with a plurality of protrusions, the plurality of protrusions configured to be received within an aperture of the external coil; and an anchoring element configured to be secured to the garment to secure the external coil to the garment.

### FIELD

The present technology is generally related to an alignment mechanism for an alignment system for use with an implantable medical device system that has the transfer of energy from a power source outside the body to an implanted medical device inside the body.

### BACKGROUND

Mechanical circulatory support devices, including ventricular assist devices (“VADs”), are used for people that have weakened hearts and/or heart failure. VADs can aid the heart's natural pumping to increase the amount of blood flow throughout the body. VADs require various external and internal components to work together to run the system so that blood is received from the patient's heart and the VAD pumps the blood back into arterial circulation. When the external and internal components of the VAD work together properly, an individual with a weakened heart and/or advanced heart failure can lead a more active and fuller life.

A transcutaneous energy transfer system (“TETS”) may be used to power implantable devices such as the VADs. Generally, a TETS can transfer energy from an external transmission coil to a receiving coil that is implanted under the skin. A TETS may be used to supplement, replace, or charge an implanted power source such as a rechargeable battery.

Some of the components that are used in the VADs include an external transmission coil and an implanted receiving coil. Various components must be properly aligned to prevent an interruption of the operation of the VADs. Movements of a patient can cause the misalignment of components from the VADs and/or the TETS which may cause the system to malfunction and in certain instances fail when power is not being transferred into the system.

### SUMMARY

The techniques of this disclosure generally relate to an attachment mechanism securable to a garment for a wireless energy transfer system, including wearable medical devices that use a transcutaneous energy transfer system (“TETS”), that require the alignment of certain external and internal components to continually facilitate the effective transfer of energy. In one aspect, the attachment mechanism securable to a garment for holding an external coil in a predetermined location, the attachment mechanism may comprise a body with a plurality of protrusions, the plurality of protrusions configured to be received within the external coil; and an anchoring element configured to be secured to the garment to secure the external coil to the garment.

In another aspect, the body has a first side and a second side opposite the first side, the first and the second sides being parallel, the plurality of protrusions being disposed circumferentially on the first side in a plane that is orthogonal to the first side.

In another aspect, the body has a central aperture, the plurality of protrusions surrounding the aperture.

In another aspect, each protrusion of the plurality of protrusions is sized to extend through at least a portion of the external coil.

In another aspect, each protrusion in the plurality of protrusions has a ridge and a protuberance, each ridge and protuberance being configured to releasably engage with the external coil.

In another aspect, each protrusion has a base portion and a tip portion, the base portion of each protrusion being permanently affixed to the body.

In another aspect, the attachment mechanism further comprises a removeable element, the removable element having at least one aperture that is sized to receive the plurality of protrusions.

In another aspect, the anchoring element has at least one strap that is configured to be secured to the garment.

In another aspect, the at least one strap is releasably secured to the anchoring element.

In another aspect, the at least one strap includes three separate straps.

In another aspect, the anchoring element further comprises a button, the button being configured to releasably engage with the external coil.

In another aspect, when the button is received within the external coil and the plurality of protrusions are received within the external coil, the button is configured to releasably engage the plurality of protrusions.

In another aspect, the attachment mechanism further comprises a receiving element, the receiving element having an aperture sized to receive the button and at least a portion of the receiving element being sized to be received within the external coil.

In one aspect, an attachment mechanism securable to a garment for holding an external coil in a predetermined location, the attachment mechanism comprising: a body having a first planar side and a second planar side opposite the first planar side with a second plane, the first and the second planar sides being parallel; a plurality of protrusions being disposed circumferentially on the first planar side in a plane that is orthogonal to the first planar side, the plurality of protrusions configured to be received within an aperture of the external coil, each protrusion sized to extend through at least a portion of the aperture of the coil, each protrusion in the plurality of protrusions having a ridge and a protuberance, each ridge and protuberance being configured to releasably engage with the aperture in the external coil, each protrusion having a base portion and a tip portion, the base portion of each protrusion in the plurality of protrusions being permanently affixed to the body; and a central aperture in the body, the plurality of protrusions surrounding the central aperture; a removeable element, the removable element having at least one aperture that is sized to be received within the plurality of protrusions, an anchoring element having: at least three straps configured to be secured to the garment, each strap being releasably secured to the anchoring element; a button, the button being configured to releasably engage with the aperture in the external coil, the button being sized to be received within the aperture in the external coil and the plurality of protrusions sized to be received within the aperture of the external coil, the button contacts the plurality of protrusions; and a receiving element, the receiving element having an aperture sized to receive the button and at least a portion of the receiving element being sized to be received within the aperture in the external coil.

In one aspect, an attachment mechanism securable to a garment for holding an external coil in a predetermined location, the attachment mechanism comprising: a body with a plurality of protrusions configured to be received within an aperture of the external coil; and
at least one aperture in the body.

In another aspect, the at least one aperture in the body is configured to engage with a portion of the garment.

In another aspect, the at least one aperture in the body is configured to engage with the external coil.

In another aspect, each protrusion in the plurality of protrusions has a base portion and a tip portion, the base portion of each protrusion being permanently affixed to the body.

In another aspect, the body has a first side and a second side opposite the first side, the first and the second sides being parallel, the plurality of protrusions being disposed circumferentially on the first side in a plane that is orthogonal to the first side.

In one aspect, an attachment mechanism securable to a garment for holding an external coil in a predetermined location, the attachment mechanism comprising: a plurality of apertures configured to engage with a portion of the external coil; a material section secured to the plurality of apertures; and a plurality of straps configured to be secured to the garment, the plurality of straps being secured to the material section.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a front inside of the body view of a patient with a left ventricular assist device, receiving coil, and controller fully implanted within the patient;

FIG. 2 is a front outside of the body view of the patient shown in FIG. 1 showing a battery and transmission coil coupled to the patient;

FIG. 3 is a front view of the receiving coil and controller shown in FIG. 1;

FIG. 4 is a side-view of an exemplary embodiment of an attachment mechanism;

FIG. 5 is a perspective view of the exemplary embodiment in FIG. 4;

FIG. 6 is a front view of the exemplary embodiment in FIG. 4;

FIG. 7 is a side view of the exemplary embodiment in FIG. 4;

FIG. 8 is a perspective exploded view of an exemplary embodiment of the attachment mechanism;

FIG. 9 is a perspective view of the exemplary embodiment of the attachment mechanism in FIG. 8;

FIG. 10 is a perspective view of the exemplary embodiment of the attachment mechanism in FIG. 8 with the components secured together;

FIG. 11 is a view of an exemplary embodiment of the attachment mechanism; and

FIG. 12 is a view of the exemplary embodiment of an attachment mechanism shown in FIG. 11 secured to an alignment garment.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

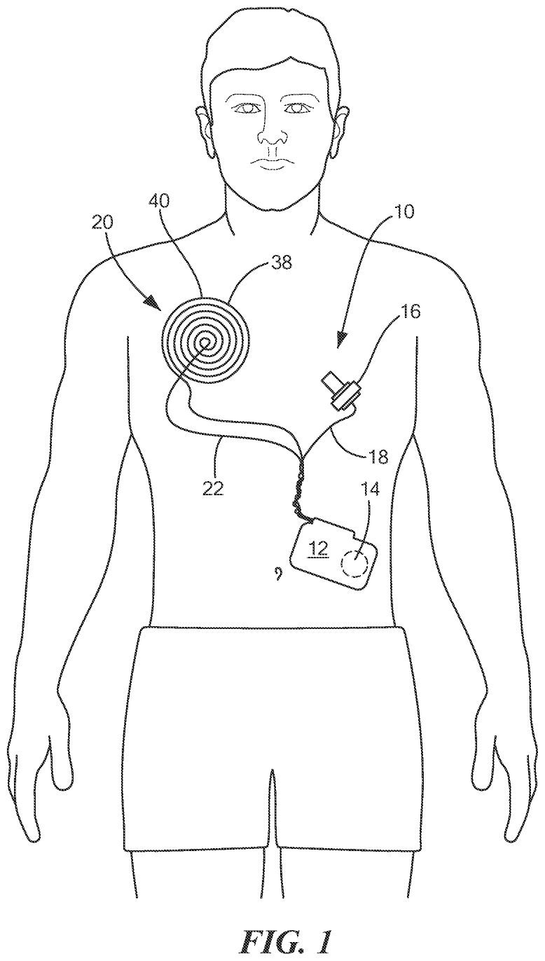

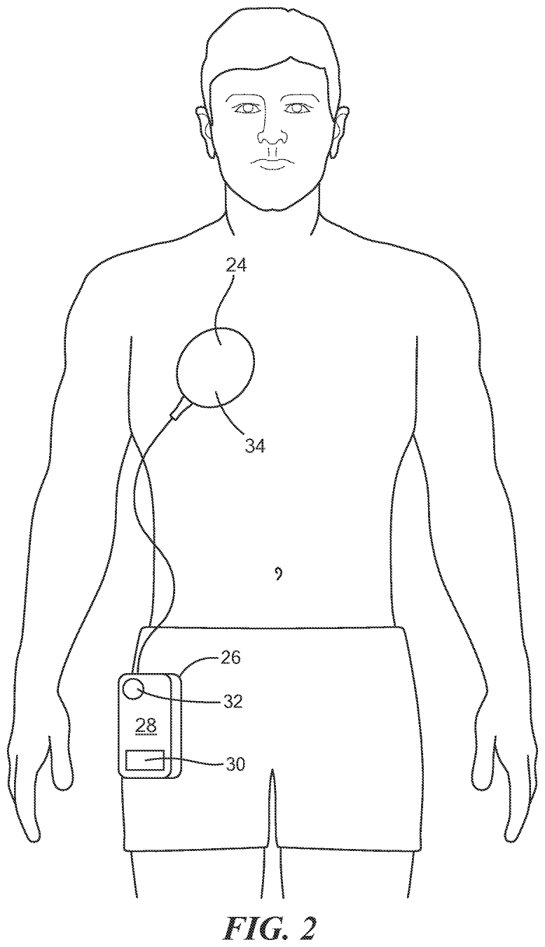

Referring now to the drawings in which like reference designators refer to like elements there is shown in FIGS. 1 and 2 an exemplary transcutaneous energy transfer system (“TETS”) constructed in accordance with the principles of the present application and designated generally as “10.” While there is discussion about a particular TETS, it will be understood that the TETS can be used for wireless energy transfer for another type of implantable medical device and can be in a variety of different configurations. The TETS 10 may be fully implantable within a patient, whether human or animal, which is to say there are no percutaneous connections between the implanted components of the TETS 10 and the components outside of the body of the patient. In the configuration shown in FIG. 1, the TETS 10 includes an internal controller 12 implanted within the body of the patient. The internal controller 12 may include a power source 14 configured to power the components of the controller and provide power to one or more implantable medical devices, for example, an implantable blood pump such as a ventricular assist device system (“VADs”) 16 implanted within the left ventricle of the patient's heart. The power source 14 may include a variety of different types of power sources including an implantable battery. The VADs 16 may include centrifugal pumps, axial pumps, or other kinds electromagnetic pumps configured to pump blood from the heart to blood vessels to circulate around the body. One such centrifugal pump is the HVAD and is shown and described in U.S. Pat. No. 7,997,854, the entirety of which is incorporated by reference. One such axial pump is the MVAD and is shown and described in U.S. Pat. No. 8,419,609. In an exemplary configuration, the VADs 16 is electrically coupled to the internal controller 12 by one or more implanted conductors 18 configured to provide power to the VADs 16, relay one or more measured feedback signals from the VADs 16, and/or provide operating instructions to the VADs 16.

Continuing to refer to FIG. 1, a receiving coil 20 may also be coupled to the internal controller 12 by, for example, one or more implanted conductors 22. In an exemplary configuration, the receiving coil 20 may be implanted subcutaneously proximate the thoracic cavity, although any subcutaneous position may be utilized for implanting the receiving coil 20. The receiving coil 20 is configured to be inductively powered through the patient's skin by a transmission coil 24 (seen in FIG. 2) disposed opposite the receiving coil 20 on the outside/exterior of the patient's body. For example, as shown in FIG. 2, a transmission coil 24 may be coupled to an external controller 26 having a power source 28, for example, a portable battery carried by the patient. The transmission coil 24 may have raised portions or be a smooth surface. The raised portions may be configured to engage with different components of the alignment system. In one configuration, the battery is configured to generate a radiofrequency signal for transmission of energy from the transmission coil 24 to the receiving coil 20. The receiving coil 20 may be configured for transcutaneous inductive communication with the transmission coil 24.

The external controller 26 may further include a display 30 configured to provide information to the patient in one or more ways including audibly, visually, and/or tacitly. Additionally, the external controller 26 may further include processing circuitry 32 which can process and transmit information. The processing circuitry 32 may perform various simple and complex operations including, but not limited to, the amplification of signals, computations, and the movement of data from one place to another. The various simple and complex operations may be performed by the processing circuitry 32 in the external controller 26. The processing circuitry 32 may include one or more processors and memory. The external controller 26 may further comprise software, which is stored in, for example, memory at the external controller 26, or stored in external memory (e.g., database, storage array, network storage device, etc.) accessible by the external controller 26. The software may be executable by the processing circuitry 32. The software may include a client application operable to provide a service to a human or non-human user via the external controller 26, with the support of a host computer.

Referring back to FIG. 2, the transmission coil 24 is optionally housed within sealed packaging 34 to protect the transmission coil 24 and is optionally secured by a garment 36, that is configured to maintain the transmission coil 24 in a fixed position for power transmission to the receiving coil 20.

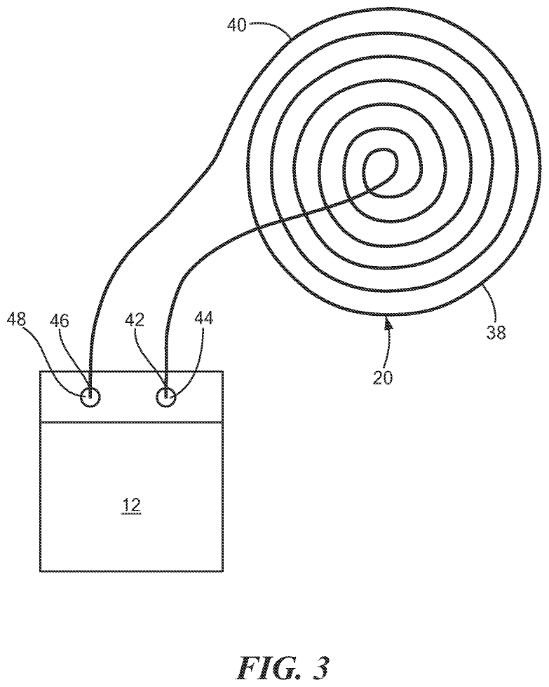

Referring now to FIGS. 1 and 3, the receiving coil 20 includes at least one conductor 38 defining a coil 40 and configured to power the VAD 16. The receiving coil 20 may be any shape or size and, for example, in one configuration, the receiving coil 20 may define a substantially planar coil defining a diameter such that is substantially co-planar with an interior surface of the dermis. The receiving coil 20 may be coupled with the internal controller 12 with a first end 42 being electrically coupled to a first coupling 44 of the internal controller 12 and a second end 46 being coupled to a second coupling 48 of the internal controller 12 such that a voltage may be applied to the receiving coil 20. Alternatively, the receiving coil 20 may be coupled with the internal controller 12 using joints or any other configuration. Although not shown, temperature sensors may be found throughout the TETS 10.

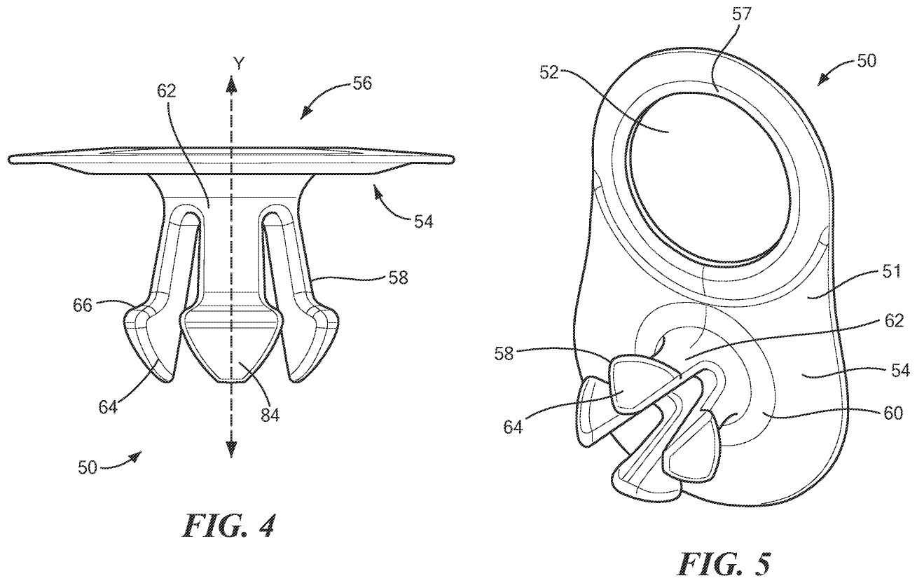

Now referring to FIGS. 4-7, there is shown an exemplary configuration of an attachment mechanism 50 which may be secured to the garment 36. The garment 36 may be a variety of different types of garments that can be worn on the body including, but not limited to, a vest, a shirt, pants, different straps configured to be secured to a portion of the body, as well as any other type of garment. The attachment mechanism 50 may be configured to permanently or releasably engage with any portion of the garment 36 to help maintain the position and alignment of certain external and internal components of a wireless energy transfer system. As a non-limiting example as shown in FIG. 1, the implanted charging component may be the receiving coil 20. It will be understood that the implanted charging component may be any other implanted component but will be generally referred to in the application as the receiving coil 20. Also, the external charging component may be any external charging component, but as a non-limiting example as shown in FIG. 2, the external charging component may be the transmission coil 24. It will be understood that the external charging component may be any other external component but will be generally referred to the in application as the transmission coil 24. To allow for energy to transfer in the TETS, the transmission coil 24 and the receiving coil 20 must be properly aligned.

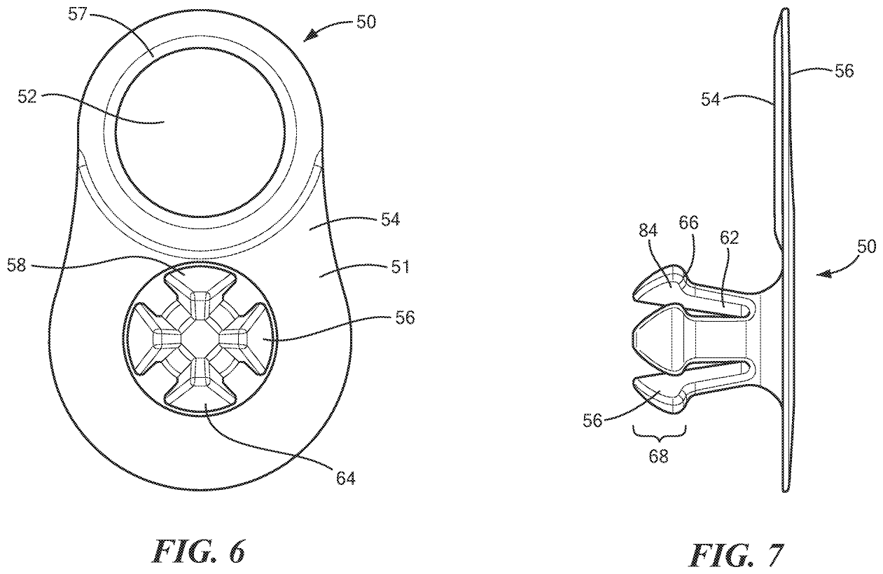

Continuing to refer to FIGS. 4-7, the attachment mechanism 50 may be used to maintain the alignment of the external charging component/transmission coil 24 over the implanted charging component/receiving coil 20 within, for example, a 2 inch tolerance along the planar and liner axes of the components for the TETS system so that power can be continually transferred effectively and efficiently. In one embodiment of the attachment mechanism 50, the attachment mechanism 50 may include a body 51 and a releasing mechanism 52. As shown in FIGS. 5 and 6, the releasing mechanism 52 may be an aperture that is configured to mate with a portion of the garment 36. The size and shape of the aperture may be based upon any corresponding portion of the garment 36 that is configured to mate with the aperture. When the releasing mechanism 52 is engaged with a portion of the garment 36, the attachment mechanism may be releasably and/or permanently secured to the garment 36. The releasing mechanism 52 may be disposed on any portion of the attachment mechanism 50 and may be a variety of different sizes and shapes depending upon the corresponding portion of the garment 36 that is configured to engage with the attachment mechanism 50. Also, the size and shape of the releasing mechanism 52 may be dependent upon the location of the receiving coil 20 in the patient's body and where the transmission coil 24 needs to be placed in relation to the receiving coil 20. In one configuration, the releasing mechanism 52 may have a substantially circular shape. The releasing mechanism 52 may be removed from the garment 36 by manually detaching the releasing mechanism 52 from the corresponding portion of the garment 36 that the releasing mechanism 52 is engaged with.

The attachment mechanism 50 may have a first side 54 and a second side 56 that is opposite the first side 54. As shown in FIGS. 4 and 7, the second side 56 of the attachment mechanism 50 may have a flat, smooth, and/or flush surface. This flat, smooth, and/or flush second side 56 can be worn against the skin for long periods of time as when the attachment mechanism 50 is secured to the garment 36, the second side 56 may come into contact with the skin and having this type of surface can prevent skin irritation and be more comfortable to wear. For example, if the patient's arm or hand brushes up against the smooth, flush, and flat surface it is less likely to cause irritation or injury to the skin. Alternatively, the second side 56 may have a rough surface which is uneven and/or a surface with different heights and can depending upon the type of releasing mechanism 52 that is part of the attachment mechanism. As shown in FIGS. 5-7, the first side 54 of the attachment mechanism 50 may include a reinforced portion 57 and which may surround the releasing mechanism 52. In one embodiment, the reinforced portion 57 may surround the releasing mechanism 52 to provide additional support around the releasing mechanism 52 and the reinforced portion 57 may be substantially circular in shape. The additional support provided by the reinforced portion 57 may allow for the securing and removal of the releasing mechanism 52 without causing wear and tear to the attachment mechanism 50. For example, in one embodiment where the releasing mechanism 52 is the aperture, the reinforced portion 57 may surround the aperture prevent breakage along the borders of the aperture.

Continuing to refer to FIGS. 4-7, body 51 with the first side 54 of the attachment mechanism 50 may include at least one protrusion 58. It will be understood that the attachment mechanism 50 may include one protrusion 58 or more than one protrusion 58. In the exemplary embodiment shown in FIGS. 4-7, there are four protrusions 58 which are configured circumferentially on the first side 54 of the attachment mechanism 50 in a plane that is orthogonal to the plane of the first side 54. If there are two or more protrusions 58, each protrusion 58 may be uniformly shaped, certain protrusions 58 may have the same shape while other protrusions have a different shape and/or size, or the protrusions 58 may all have different shapes and sizes. As shown in FIG. 5, the protrusions 58 may be secured to the first side 54 of the attachment mechanism 50. In one configuration, the protrusions 58 may be secured to the first side 54 and a raised element may be disposed around a portion of the protrusions 58. The raised element 60 in one configuration may be circular and configured to circumscribe the protrusions 58 and secure and/or reinforce a portion of the protrusions 58 to the first side 54. The raised element 60 may be a variety of different shapes and sizes and the shape and size may be based upon the shape and size of the protrusions 58 and/or the attachment mechanism 50.

In one configuration, each protrusion 58 may have a base portion 62 and a tip portion 64. The size and shape of each protrusion 58 may be configured so that each protrusion 58 can mate with the transmission coil 24. Each protrusion 58 may have a circumference or they may be square, rectangle, or any other shape that is configured to mate with the transmission coil 24. In one embodiment, the transmission coil 24 may have at least one aperture that corresponds with each protrusion 58 and the at least one aperture may be sized and shaped to receive each protrusion 58. Alternatively, the transmission coil 24 may have the sealed packaging 34 and the sealed packaging 34 may have at least one aperture that corresponds with each protrusion 58 and the at least one aperture may be sized and shaped to receive each protrusion 58. When each protrusion 58 is received within the corresponding aperture in the transmission coil 24, the attachment mechanism may be releasably and/or permanently secured to the transmission coil 24. The size and shape of each protrusion 58 may be based upon the mating mechanism in the transmission coil 24 that is sized to receive each protrusion 58.

As shown in FIGS. 4-7, the base portion 62 of the protrusion 58 may be integrated into the first side 54 of the attachment mechanism 50. Alternatively, the base 62 of the protrusion 58 may be inserted into the first side 54 of the attachment mechanism 50 and may be removable so that different protrusions 58 may be inserted into the first side 54 of the attachment mechanism 50. The tip portion 64 of the protrusions 58 may be the same size and shape as the base portion 62 or the tip portion 64 may be larger, smaller, longer, or shorter than the base portion 62. In one exemplary embodiment, the tip portion 64 of the protrusion 58 may be larger than the base portion 62. For example, as shown in FIGS. 4-5, the base portion 62 may be integrated into the first side 54 of the attachment mechanism 50 and have a size and shape at the point where the base portion 62 is integrated into the first side 54. The base portion 62 may have an increasing diameter from the first side 54 where the base portion 62 is integrated into the attachment mechanism 50 to the tip portion 64. As shown in FIG. 4, along longitudinal axis Y, the diameter of the protrusion 58 increases from the base portion 62 to the tip portion 64. The tip portion 64 of each protrusion 58 may include a ridge 66 and a protuberance 68. In one configuration the ridge 66 may define where the protuberance 68 is disposed on the tip portion 64 of the protrusion 58 and the ridge 66 may have the largest diameter on each protrusion 58. The protuberance 68 may be any size and shape and configured to mate with the corresponding apertures that correspond with each protrusion in the transmission coil 24. The protrusions 76 may be movable in different directions so that they can be mated with and releasably secured to the transmission coil 24 and will depend upon the mating mechanism with the transmission coil 24.

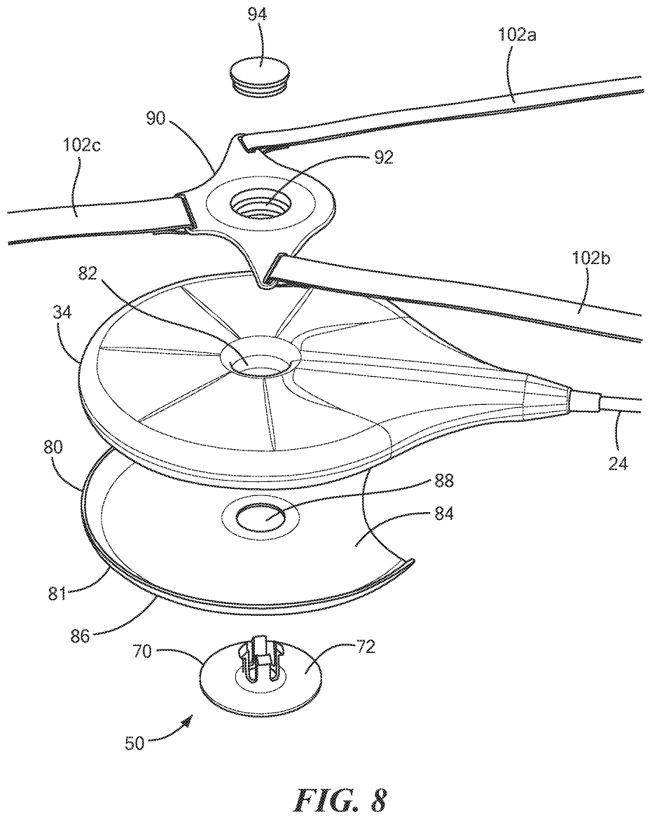

Now referring to FIGS. 8-10 where another exemplary embodiment of the attachment mechanism 50 which can be secured to the garment 36 is shown. In the embodiment shown in FIG. 8, the attachment mechanism 50 may have different components in this configuration and may include an alignment element 70, the alignment element 70 may have a first side 72 and a second side 74 opposite the first side 72 with at least one protrusion 76 being secured to the first side 72. The first second side 74 of the alignment element 70 may have silicon or other material and/or fabric to prevent movement of the attachment mechanism 50 and the receiving coil 24 when they are secured together with the garment 36 and against or near the skin. It will be understood that the at least one protrusion 76 may be the same size as shape as those shown in FIGS. 4-7 or may have a different shape and/or configuration. As shown in FIG. 8, in one configuration, there may be four protrusions 76 secured to the first side 72 of the alignment element 70. The four protrusions 76 may be secured around an aperture 78 within the alignment element 70. In one configuration, the aperture 78 may be circular and the four protrusions 76 may be evenly disposed around the circular aperture 78.

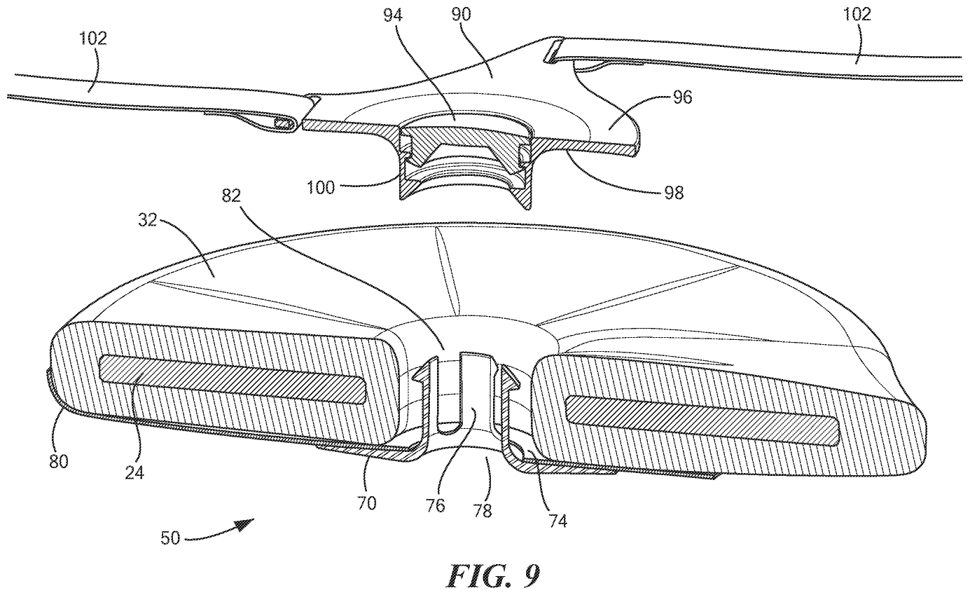

The attachment mechanism 50 may also include a removable element 80. The removable element 80 may be sized and shaped to fit around the transmission coil 24. In one configuration, the transmission coil 24 may be disposed within the sealed packaging 34 and the sealed packaging 34 may be disposed around the transmission coil 24 to provide protection around the transmission coil 24 and to further separate the transmission coil from the skin of the body. In one configuration, the sealed packaging 34 may have at least one aperture 82. As shown in FIGS. 8 and 9, the at least one aperture 82 in the sealed packaging 34 may be disposed through the transmission coil so that different components of the attachment mechanism 50 may be secured together with the sealed packaging 34. The removable element 80 may have a first side 84 that is sized and shaped to be in direct contact with the sealed packaging 34 on the transmission coil 24. As shown in one embodiment in FIG. 8, the first side 84 of the removable element 80 may have a planar portion that is sized to receive a corresponding planar portion of sealed packaging 34 on the transmission coil 24 as well as a raised portion that is configured to be secured around the edges of the sealed packaging 34 on the transmission coil 24. The removable element 80 may have a second side 86 opposite the first side that is configured to come into contact with the alignment element 70. The size and shape and the removeable element 80 may be based upon the size and shape of the sealed packaging 34 around the transmission coil 24. As shown in FIG. 8, the planar portion of the removable element 80 may be substantially circular and the raised portion may also be substantially circular to be secured around the edges of the transmission coil 24. The removable element 80 may also have an aperture 88 that is configured to correspond with the aperture 82 in the sealed packaging 34. It will be understood that the transmission coil 24 may not have the sealed packaging 34 so that the removable element 80 may be directly secured to the transmission coil 24 as well. Alternatively, the removable element 80 may not be used and the alignment element 70 may be secured to the sealed packaging 34 without the removable element 80. In this configuration, the alignment element 70 may include the at least one protrusion 76 and the at least one protrusion 76 may be configured to correspond with the aperture 82 in the sealed packaging 34. The second side 74 of the alignment element 70 may be smooth and may contain an aperture in the center with the at least one protrusion 76 surrounding the aperture. The second side 74 of the alignment element 70 may come into contact with the skin and not cause irritation. The alignment element 70 may also have slits or other apertures (not shown) that are configured in a variety of different patterns and may be used to secure the alignment element 70 to a belt, garment, or article of clothing to help keep the alignment element 70 in place on the body. In another embodiment, the at least one protrusion 76 may be secured to at portion of the sealed packaging 34. In this configuration, the at least one protrusion 76 may be secured around the aperture 82 in the sealed packaging 34. The at least one protrusion 76 may also be a part of and/or secured to the anchoring element 90.

As shown in FIG. 8, the attachment mechanism 50 may also include an anchoring element 90. The anchoring element 90 may include a receiving element 92 and in one configuration the receiving element 92 may be an aperture. The attachment mechanism 50 may also include a releasable mechanism 94 and the releasable mechanism 94 may be a button. It will be understood that the releasable mechanism 94 may be referred to generally as a button but can refer to any type of releasable mechanism. In one configuration, the button 94 may be sized and shaped to be received within at least a portion of the anchoring element 90 and specifically, as shown in FIG. 8, within the receiving element 92 of the anchoring element 90. As shown in FIG. 9, the anchoring element 90 may have a first side 96 and a second side 98 opposite the first side 96. The first side 96 of the anchoring element 90 may be planar and smooth so that if the skin touches this portion of the anchoring element 90 it will not cause irritation or harm to the skin. The second side 98 of the anchoring element 90 may not be planar and, as shown in FIG. 9, in one embodiment may include an extended portion 100. The extended portion 100 may correspond to the size and shape of the receiving element 92 of the anchoring element 90. As a non-limiting example, the extended portion 100 may form a lip around the receiving element 92 such that the extended portion 100 is sized to be received within the aperture 82 in the sealed packaging 34 and may also be configured to be secured to the protrusions 76 in the alignment element 70. Alternatively, the extended portion 100 may include a ridge such that a portion of the releasing mechanism 94 may be sized to be releasably received within the ridge. The releasable mechanism 94 may be compressed against the ridge and when the compression occurs the releasable mechanism 94 may be released from ridge within the extended portion 100. Additionally, when the releasable mechanism 94 is secured within the extended portion 100 and the at least one protrusion 76 is also secured within the extended portion 100, the releasable mechanism 94 and the at least one protrusion 76 may be in contact with one another or in close proximity to one another. When the releasable mechanism 94 is depressed into the extended portion 100, the at least one protrusion 76 may be compressed such that the at least one protrusion 76 is no longer in contact with the ridge within the extended portion and the at least one protrusion 76 may be released from the extended section allowing for the removal of the alignment element 70. In an alternative embodiment (not shown), the releasable mechanism 94 may have a protuberance that is sized so that the protuberance may be used to hold onto the releasable mechanism 94 and can secure the releasable mechanism 94 into the receiving element 92 or it may be used to remove the releasable mechanism 94 from the receiving element 92. In one embodiment, the at least one protrusion 76 may be secured to a portion of the anchoring element 92 and the at least one protrusion 76 may be sized to be received within at least a portion of the receiving element 92. When the at least one protrusion 76 is received within the receiving element 92, the at least one protrusion 76 may extend into the extended portion 100 and be sized to extend beyond the ridge within the extended portion 100. In one embodiment, the releasable mechanism 94 could be depressed to apply force to the at least one protrusion 76 and move the at least one protrusion 76 away from the ridge in the extended portion 100.

Continuing to refer to FIGS. 8-10, the anchoring element 90 may also have at least one strap 102. As shown in the configuration in FIG. 8, the anchoring element 90 may include three straps 102a, 102b, and 102c. It will be understood that the anchoring element 90 may include any number straps which are configured to engage with at least a portion of the garment 36. The anchoring element 90 may also not have any straps 102 and may configured to engage with a portion of the garment 36 with a different mechanism as well. The at least one strap 102 may be permanently secured and/or releasably secured to a portion of the garment 36. The manner and placing of at least one strap 102 may depend upon where the transmission coil 24 and the corresponding receiving coil 20 are to be disposed so that conductive charging can take place.

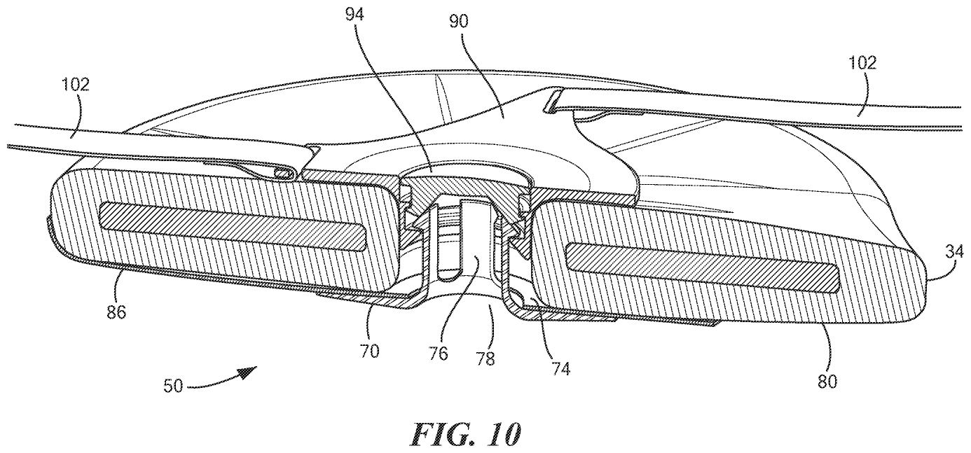

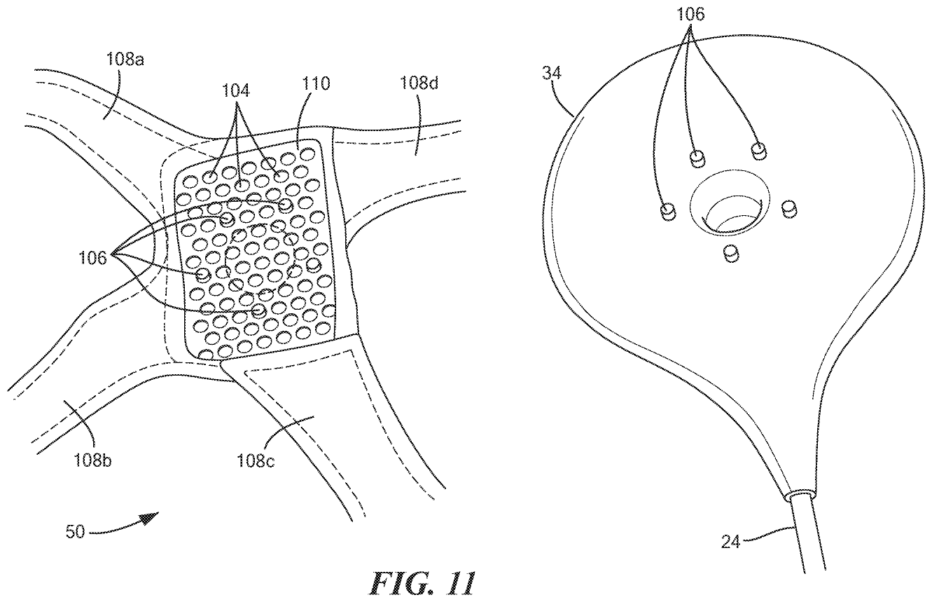

Now referring to FIGS. 9 and 10, the releasable mechanism 94, the button as shown, may be sized and shaped to be received within the receiving element 92. When the releasable mechanism 94 is received within the receiving element 92, the first side 96 of the anchoring element 90 may be smooth and flush so that if this surface comes into contact with the skin it does not cause irritation or injury. When the releasable mechanism 94 is disposed within the receiving element 92, the releasable mechanism 94 may contact at least a portion of the protrusions 76. As shown in FIG. 10, a portion of the releasable mechanism 94 may be in contact with the tip portion of each protrusion 76. If the releasable mechanism 94 is the button, the button 94 may be pressed and the depressing of the button 94 in the receiving element 92 may release the protrusions 76 so that the various components of the attachment mechanism 50 can be unsecured and separated. In a non-limiting example, the releasable mechanism 94 may be secured within the receiving element 92 of the anchoring element 90. The anchoring element 90 may then be secured within the aperture 82 of the sealed packaging 34 and the removable element 80 may also be secured to the sealed packaging 34 of the transmission coil 24. The protrusions 76 of the alignment element 70 may be received within the aperture 88 in the removable element as well as within the aperture 82 of the sealed packaging 34. Pressing the releasable mechanism 94 down into the receiving element 92 can cause release of the protrusions 76 from in the alignment element 70 and the receiving element 92 and the anchoring element can be separated from the removable element 80 and the alignment element 70 may be separated as well. This can allow for the removal of the attachment mechanism 50 from the transmission coil 24.

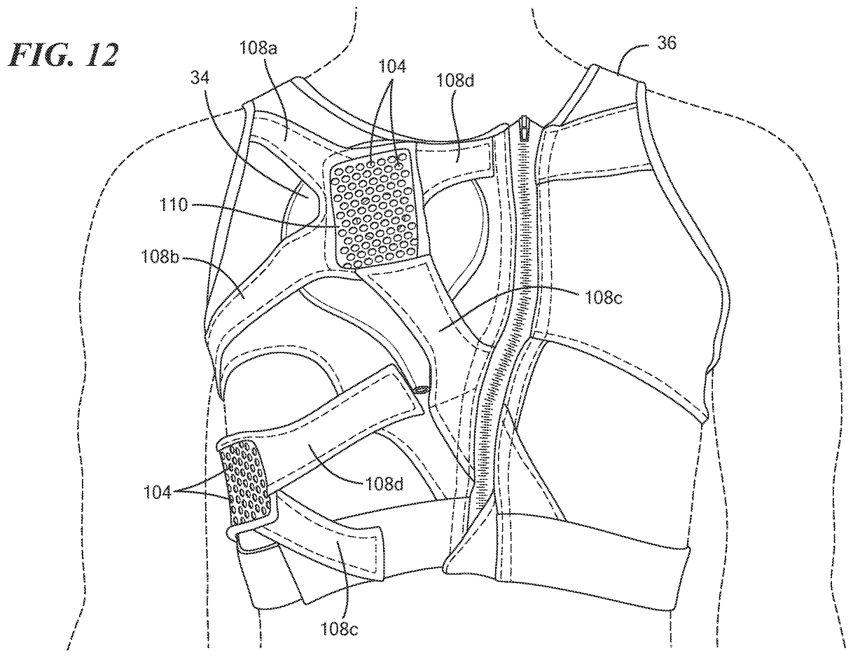

Now referring to FIGS. 11 and 12, another exemplary embodiment of an attachment mechanism 50 is shown. In this embodiment, the attachment mechanism 50 has a at least one aperture 104, which can include a plurality of apertures 104 that can be a variety of different shapes and sizes. The shape and size of the apertures 104 are sized and shaped to correspond with the size and shape of at least one protrusion 106 on the sealed packaging 34. In one configuration, the plurality of apertures 104 may be configured in a honeycomb type-pattern, as shown in FIG. 11, such that a plurality of protrusions 106 maybe secured into the plurality of apertures 104. The plurality of apertures 104 in the honeycomb type-pattern may be spaced between approximately 0.1-5 inches apart. The at least one protrusion 106 may be permanently and/or releasably secured to the sealed packaging 34 of the transmission coil 24. Alternatively, the at least one protrusion 106 may be permanently and/or releasably secured to the transmission coil 24. The plurality of apertures 104 allows the at least one protrusion 106 to be easily removed and secured to different apertures 104 so that the transmission coil 24 can be easily moved into different positions for comfort and to align with the receiving coil 20. In one embodiment, there may be only one protrusion 106 on the sealed packaging 34 which my allow for the rotation of the sealed packaging 34 while it is secured into the correspond aperture 104. In one embodiment with only one protrusion 106 on the sealed packaging 34, the at least one protrusion 106 may be in center of the sealed packaging 34 and the sealed packaging 34 may not include an aperture 82. In an alternative embodiment, and as shown in FIG. 11, the sealed packaging 34 may have five protrusions 106 which may allow for less movement of the sealed packaging 34 when it is secured into the aperture 104. It will be understood that there may be one protrusion 106 and in alternative embodiments there may be more than one protrusion 106 to secure the sealed packaging 34 in the plurality of apertures 104. The number of apertures 104 may correspond to the number of protrusions 106 or there may be a fixed number of apertures 104.

Continuing to refer to FIGS. 11 and 12, the attachment mechanism 50 may also include a plurality of straps 108. In the configuration as shown in FIGS. 11 and 12, the exemplary embodiment includes four straps 108a, 108b, 108c, and 108d. The plurality of straps 108 may be in a variety of different configurations and depend upon where the attachment mechanism 50 is to be secured to the garment 36. The reinforcement of the four straps 108a, 108b, 108c, and 108d can concentrate tension through a particular portion of the attachment mechanism 50 to help deform the transmission coil 24 and the attachment mechanism 50 to the shape of the body. The four straps 108a, 108b, 108c, and 108d may also be pretensioned with a particular tension to secure the attachment mechanism 50 and the receiving coil 24 to the body. The attachment mechanism 50 may also have a material section 110 that surrounds the plurality of apertures 104. The material section 110 may be made from a thin polymer sheet fabric to minimize the bulkiness of the attachment mechanism 50 and may be in a variety of different shapes and sizes. The plurality of straps 108 may be secured to the material section 110 and the plurality of straps 108 may be secured to a portion of the garment 36.

As shown in FIG. 12, the attachment mechanism 50 is shown as secured to the garment 36 with the transmission coil 24 being secured to the pectoral region of the body. In this embodiment, the plurality of straps 108 are secured to different portions of the garment 36. The transmission coil is releasably secured to the attachment mechanism 50 with the at least one protrusion 106 being secured in the corresponding aperture 104 so that the transmission coil 24 can be easily removed from the attachment mechanism 50 and the garment 36. The garment 36 may have a second attachment mechanism 50 as shown in FIG. 12 that is secured to the side of the patient's body. The garment 36 may include any number of attachment mechanisms 50 include one attachment mechanism 50 or more than one attachment mechanism 50.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

1. An attachment mechanism securable to a garment for holding an external coil in a predetermined location, the attachment mechanism comprising:
a body with a plurality of protrusions, the plurality of protrusions configured to be received within the external coil; and
an anchoring element configured to be secured to the garment to secure the external coil to the garment.

2. The attachment mechanism of claim 1, wherein the body has a first side and a second side opposite the first side, the plurality of protrusions being disposed circumferentially on the first side.

3. The attachment mechanism of claim 2, wherein the body has an aperture, the plurality of protrusions surrounding the aperture.

4. The attachment mechanism of claim 1, wherein each protrusion of the plurality of protrusions is sized to extend into at least a portion of the external coil.

5. The attachment mechanism of claim 4, wherein each protrusion of the plurality of protrusions has a ridge and a protuberance, each ridge and protuberance being configured to releasably engage with the external coil.

6. The attachment mechanism of claim 5, wherein each protrusion has a base portion and a tip portion, the base portion of each protrusion being permanently affixed to the body.

7. The attachment mechanism of claim 1, further comprising a removeable element, the removable element having at least one aperture that is sized to receive the plurality of protrusions.

8. The attachment mechanism of claim 1, wherein the anchoring element has at least one strap that is configured to be secured to the garment.

9. The attachment mechanism of claim 8, wherein the at least one strap is releasably secured to the anchoring element.

10. The attachment mechanism of claim 9, wherein the at least one strap includes three separate straps.

11. The attachment mechanism of claim 9, wherein the anchoring element further comprises a button, the button being configured to releasably engage with the external coil.

12. The attachment mechanism of claim 11, wherein when the button is received within a portion of the external coil and the plurality of protrusions are received within the external coil, the button is configured to releasably engage the plurality of protrusions.

13. An attachment mechanism securable to a garment for holding an external coil in a predetermined location, the attachment mechanism comprising:
a body having a first planar side and a second planar side opposite the first planar side, the first and the second planar sides being parallel, wherein the body defines a central aperture;
a plurality of protrusions being disposed circumferentially on the first planar side, the plurality of protrusions configured to be received within an aperture of the external coil, each protrusion of the plurality of protrusions sized to extend through at least a portion of the aperture of the external coil, each protrusion of the plurality of protrusions having a ridge and a protuberance, each ridge and protuberance being configured to releasably engage with the aperture, and each protrusion of the plurality of protrusions having a base portion and a tip portion, the base portion of each protrusion of the plurality of protrusions being permanently affixed to the body,
wherein the plurality of protrusions surrounding the central aperture;
a removeable element, the removable element having at least one aperture that is sized to receive the plurality of protrusions,
an anchoring element including:
at least three straps configured to be secured to the garment;
a button, the button being configured to releasably engage with the aperture of the external coil, the button being sized to be received within the aperture of the external coil and the plurality of protrusions sized to be received within the aperture of the external coil, the button contacts the plurality of protrusions; and
a receiving element, the receiving element having an aperture sized to receive the button and at least a portion of the receiving element being sized to be received within the aperture of the external coil.

14-15. (canceled)

16. The attachment mechanism of claim 12, further comprising a receiving element having an aperture sized to receive the button, at least a portion of the receiving element being sized to be received within the external coil.

17. An assembly comprising:
a garment configured to be worn on a body of a patient; and
an attachment mechanism securable to the garment to hold an external coil in a predetermined location, the attachment mechanism comprising:
a body defining at least one body aperture; and
a plurality of protrusions extending from the body, the plurality of protrusions being configured to be received within a coil aperture of the external coil.

18. The assembly of claim 17, wherein the at least one body aperture is configured to engage with a portion of the garment.

19. The assembly of claim 17, wherein the at least one body aperture is configured to engage with the external coil.

20. The assembly of claim 17, wherein each protrusion of the plurality of protrusions has a base portion and a tip portion, the base portion of each protrusion being permanently affixed to the body.

21. The assembly of claim 17, wherein the body has a first side and a second side opposite the first side, the first side and the second side being parallel, and the plurality of protrusions being disposed circumferentially on the first side.

22. The assembly of claim 17, further comprising the external coil.
